(19) United States
(12) Patent Application Publication
(10) Pub. No.: US 2024/0398229 A1
(43) Pub. Date: Dec. 5, 2024

(54) SYSTEM AND METHOD FOR AUTOMATIC CALIBRATION AND ALIGNMENT OF FUNDUS CAMERA DEVICE

(71) Applicant: Oivi AS, Oslo (NO)

(72) Inventors: Aadarsh Mehndiratta, Oslo (NO); Ahamed Khaleel Udyawar Abdul, Bangalore (IN); Ankit Maurya, Bangalore (IN); Anders Eikenes, Bærum, Akershus (NO); Hans Einar Overjordet, Asker, Akershus (NO); Sarthak Prakash, Oslo (NO); Jukka Alasirnio, Oulu (FI)

(21) Appl. No.: 18/701,239

(22) PCT Filed: Oct. 12, 2022
(86) PCT No.: PCT/GB2022/052599

§ 371 Date: Apr. 14, 2024

(30) Foreign Application Priority Data
Oct. 14, 2021 (IN) 202141046889

(51) Int. Cl. A61B 3/15

(52) U.S. Cl. CPC A61B 3/152; A61B 2560/0223

(57) ABSTRACT

A method of validating alignment of an image sensor of a camera and an illumination projection for use in a system for automatically aligning a camera device which includes the camera, wherein the camera has an image sensor wherein a centre of the image sensor is identified; and the camera device includes an illumination source wherein a centre of an illumination projection is identified, and the camera device also includes a stereo camera in addition to the camera, the method comprising: calculating error between the centre of the image sensor and the centre of the illumination projection and validating alignment of the image sensor and the illumination projection if the error is within a predefined threshold.

### REFERENCE

This application claims the benefit of the filing date of Indian Complete application No. 202141046889, filed Oct. 14, 2021, the disclosures of which are hereby incorporated by reference herein.

### FIELD

Various embodiments of the disclosure relate to methods, devices and systems for automatic calibration and alignment of a fundus camera. More particularly, the present invention relates to a method, a device and a system for calibration of a stereo camera, movable platform, positioning based on illumination projection of a fundus camera device and a fast validation thereof.

### BACKGROUND

A fundus camera is a device designed to capture a fundus image i.e., an image of the rear of the eye to capture features such as retina, optic disc, macula, etc. Fundus cameras are primarily used by professionals such as optometrists, ophthalmologists, etc. for diagnosis and imaging of the fundus. A fundus camera device typically requires a highly trained operator to align the fundus camera with the pupil of the eye.

Generally, a fundus camera device set-up involves a fundus camera and other parts such as a movable platform, illumination source such as flash, and alignment sensors. A movable platform can be used to manoeuvre the fundus camera for achieving the ideal position for capturing the fundus image. Movable platforms can have movements in various axes linearly or rotationally. For example, X-Y-Z movement, pan-tilt-Z movements, one axis at a time or several axes simultaneously.

Camera calibration refers to the process of determining parameters of the camera setup estimating the intrinsic and extrinsic properties of the camera. Intrinsic parameters, in general, help to determine which incoming light ray is associated with which pixel in the image sensor. Intrinsic parameters may include but are not limited to properties of a single camera such as its focal length, axis skew, distortion, image centre, etc. Extrinsic parameters describe its position and orientation in the real-world, positional relationships between multiple cameras, translational and rotational offsets, etc.

A stereo camera generally refers to a special set-up of two cameras that are able to capture three-dimensional images. Generally, the calibration of stereo cameras is performed using a checkerboard as the calibration target.

Further, there are high chances that the alignment or calibration of the fundus camera or other parts of the system may get impacted due to harsh transportation conditions, the operational environment, etc. Any change in the alignment or calibration of the fundus camera or any parts of the device may affect the quality of the fundus image.

Therefore, there is a requirement to determine the working condition of the fundus camera and other parts of the device prior to using it for any fundus imaging. The limitations and disadvantages of conventional and traditional approaches will become apparent to one of skill in the art, through comparison of described systems with some aspects of the present disclosure, as set forth in the remainder of the present application and with reference to the drawings.

### SUMMARY

Disclosed is a method of validating alignment of an image sensor of a camera and an illumination projection as claimed in claim 1, and corresponding system and computer program.

A fundus camera device and automatic alignment of the device are described. In one embodiment, the automatic alignment of the fundus camera device comprising an optical system, a fundus camera, a stereo camera, an illumination source and a movable platform is described. The stereo camera, placed at the distal end of the fundus camera device, comprises a left camera and a right camera. Calibration of the stereo camera includes capturing images of the multi-planar calibration target. Each plane of the multi-planar calibration target comprises multiple fiducial markers. The calibration of the optimal position of the fundus camera device for capturing a fundus image includes calibration using the illumination source wherein the illumination source may be used to illuminate the eye calibration target. On performing the calibration of the stereo camera and the optimal position of the fundus camera device, the fundus camera device can be automatically aligned.

The disclosure further includes the calibration of a movable platform. The variation in the movement between the axes of the movable platform and the axes of the stereo camera is used to ascertain the error. Once the error is ascertained, the average error for all the axes is calculated. To minimize the average error, the axes of the stereo camera are virtually rotated to match the axes of the movable platform.

Additionally, the disclosure includes a fast validation of the fundus camera device prior to use. Using the stereo camera, an image of the multi-planar calibration target is captured and the average of the reprojection error is calculated for all the detected fiducial markers in the captured image. Based on the calculated average of reprojection error, it is validated if the average error is within a predefined threshold. Further, the fundus camera device is positioned such that the eye calibration target is at the optimal coordinates. The illumination source is then turned on and made to illuminate the eye calibration target. The error between the saved illumination projection properties of a correctly calibrated fundus camera device and the illumination projection properties observed in this validation is calculated. Based on the calculated error in projection properties, it is validated if the error is within a predefined threshold.

In another embodiment, the alignment between the image sensor of the fundus camera and the projection of the illumination source is validated. The validation includes calculating the error between the identified centre of the illumination projection and the centre of the fundus camera image. Once the error is calculated, it is validated if the error is within a predefined threshold. To calculate the error between the centre of the illumination projection and the centre of the fundus image, the eye calibration target and stereo camera may also be used in the process.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

The drawings illustrate some examples of embodiments disclosed herein and do not limit the invention. It should be noted that the figures are not drawn to scale and that elements of similar structures or functions are represented by reference numerals throughout the figures.

FIG. 1 illustrates a schematic block diagram of an exemplary system for automatic alignment of a fundus camera device according to an embodiment of the present invention.

FIG. 2(A) and FIG. 2(B) depict an exemplary projection view and front view of a fundus camera device respectively according to an embodiment of the present invention.

FIG. 3 shows an exemplary multi-planar calibration target according to an embodiment of the present invention.

FIG. 4 illustrates images of a calibration target captured by the left and the right camera of stereo camera according to an embodiment of the present invention.

FIG. 5(A), FIG. 5(B), FIG. 5(C), FIG. 5(D), and FIG. 5(E) show exemplary visualizations of reprojection error according to an embodiment of the present invention.

FIG. 6(A), and FIG. 6(B) depicts an exemplary axis system of a stereo camera and a movable platform according to an embodiment of the present invention.

FIG. 7 is a flowchart illustrating a process for automatically calibrating a fundus camera device according to an embodiment of the present invention.

FIG. 8 is a flowchart illustrating a process for calibration of a stereo camera according to an embodiment of the present invention.

FIG. 9 is a flowchart illustrating a process for movement calibration of a movable platform and a stereo camera according to an embodiment of the present invention.

FIG. 10 is a flowchart illustrating a process for calculation of reprojection error according to an embodiment of the present invention.

FIG. 11 shows a flowchart illustrating a process for calibration of the position of the fundus camera device using an illumination source according to an embodiment of the present invention.

FIG. 12 illustrates a flowchart of an exemplary fast validation process according to an embodiment of the present invention;

FIG. 13 illustrates a flowchart of an exemplary validation process for the alignment of the centre of the image sensor of the fundus camera according to an embodiment of the present invention.

FIG. 14 illustrates a flowchart of an exemplary alternate way of validating the alignment of the centre of the image sensor of the fundus camera according to an embodiment of the present invention.

FIG. 15 (A) illustrates an exemplary illumination path of the fundus camera using an optical system according to an embodiment of the present invention.

FIG. 15 (A) illustrates an exemplary imaging path of the fundus camera using an optical system according to an embodiment of the present invention.

FIG. 16(A), FIG. 16 (B), FIG. 16(C) illustrate the exemplary effect of positioning of the fundus camera device to an embodiment of the present invention.

### DETAILED DESCRIPTION

Various embodiments of the present invention will be described in detail with reference to the drawings, which are provided as illustrative examples of the invention to enable those skilled in the art to practice the invention. The figures and the examples below are not meant to limit the scope of the present invention. Where certain elements of the present invention may be partially or fully implemented using known components or processes, only those portions of such known components or processes that are necessary for an understanding of the present invention will be described, and the detailed descriptions of other portions of such known components or processes will be omitted so as not to obscure the invention. Further, various embodiments include present and future known equivalents to the components referred to herein by way of illustration.

In one embodiment of the invention, the present invention relates to a method and a system for the automatic alignment of a fundus camera device. A fundus camera device may include a stereo camera, a fundus camera, an illumination source, and a movable platform. The motors, sensors for detection, an electronic component to operate and capture images may also be employed to perform respective functions.

Another embodiment of the invention relates to the calibration of a stereo camera using a multi-planar calibration target (also referred to as MCT). In another embodiment of the invention, the present invention discloses movement calibration of a movable platform. In yet another embodiment, an illumination source is used to calibrate the position of the fundus camera device relative to the location of the eye calibration target (also referred to as ECT). In another embodiment, the alignment between the centre of the illumination projection and the centre of the image sensor of the fundus camera is validated. In yet another embodiment, the present invention relates to validation of the fundus camera device prior to use and automatic alignment of the fundus camera thereof.

In one embodiment of the invention, calibration of the stereo camera is performed using a calibration target. A stereo camera may comprise a set-up of two or more cameras which may be placed towards the distal end of the fundus camera. Calibration of the stereo camera, for example, involves taking an image of a calibration target using a left camera and a right camera of the stereo camera. The calibration target may be a multi-planar target comprising multiple planes. Each plane of the MCT may be embedded with several fiducial markers.

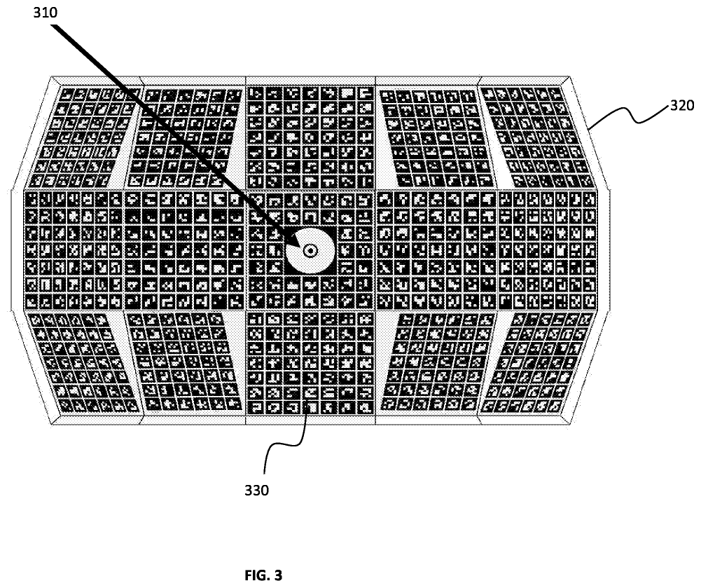

In the present invention, as an example, individually identifiable markers are used as fiducial markers in MCT without any repetition. This allows identifying and grouping the fiducial markers based on the plane they belong to and also identify their pre-known positions in their plane. Additionally, each marker may be mapped with one object point and one or more respective image points based on the predefined position of the marker. Further, one of the planes of the MCT may include an eye calibration target (also referred to as ECT). In one embodiment of the invention, the ECT is located in the centre of the central plane of the MCT. Further, the size of the ECT may approximately be equal to the size of the pupil of the eye, further comprising two smaller circles within the pupil portion as represented by 310 of FIG. 3.

The multiple images of the planes of the MCT may be captured in one or more imaging instances using a camera. The multiple images may be captured by moving the fundus device or the calibration target. For example, an image of the MCT may be captured using the left camera and the right camera of the stereo camera. The left camera and the right camera may capture a full view or a partial view of the planes. For all the captured images, the images are processed through an identifier to identify each uniquely identifiable marker visible in the plane. It will be apparent to a person skilled that libraries such as OpenCV may be used to implement identification for markers such as Aruco markers. The extracted unique identifiers and associated information are stored in a database of the computing system. The associated information of a marker may include an ID of the marker, image points of the marker, object points of the marker etc. The identified markers are grouped based on the planes they belong to. After separating the object points for the respective plane, a corresponding mapping list may be defined with their known real-world coordinates in 2-D wherein the depth will be zero since all the object points belong to the same plane. Further, object coordinates may be defined as 3-D coordinates of the object points with one of the axes set to zero.

Subsequently, the mapped image points and object points may be stored in the database for any further processing, such as running world-coordinates reprojection error calculations. For the captured image, each image may be of different quality. In a few of the images, a few markers may not be detected due to certain constraints such as focus, field of view, mechanical limitations etc. To allow for maximum adaptability across versions of the fundus camera device, validation may be performed. While performing the validation, the minimum number of unique identifiers in each group of the planes may be determined. If the minimum number of the unique identifiers exists within the threshold value, the validation is considered successful for the plane. Once the validation is performed successfully, the intrinsic properties of the left camera and the right camera are calibrated. The calibration of intrinsic properties involves processing the image points and the object points for each of the planes. Intrinsic properties may include but are not limited to properties of a single camera such as its focal length, axis skew, distortion, image centre, etc. Thereafter, the calibration of extrinsic properties of the stereo camera may be performed. The calibration of extrinsic properties may involve processing the image points and the object points for each of the planes using only mutually identified markers in both left and right cameras. Extrinsic parameters may include its position and orientation in the real world, axis system of the stereo camera, positional relationships between multiple cameras, translational and rotational offsets etc. Thereafter, all the properties related to the intrinsic and extrinsic calibrations are stored in a database for further processing such as additions, modifications, or validations and the like. It will be apparent to a person skilled that the libraries for camera calibrations such as OpenCV may be used for intrinsic and extrinsic calibration of cameras, which may use algorithms such as mentioned by Zhang et. al.

In one embodiment of the invention, the movement of the movable platform may be calibrated. The movement calibration of the movable platform may ensure that the movable platform moves as instructed by the stereo camera. In other words, if the stereo camera instructs the movable platform to move by, say (x,y,z), the platform will move exactly by (x,y,z) in the axis system of the stereo camera.

Initially, the axes of the movable platform and the axes of the stereo camera are identified. On selecting a particular axis, any variation in movement in this axis of the stereo camera and the movable platform may be determined. Variation in movement may be calculated by moving the platform by a fixed distance, say x′ in X axis, while simultaneously capturing images of the MCT using the stereo camera. During this process, the stereo camera is used to calculate the shift in the position of markers in its own x axis. Let's assume the movement calculated by the stereo camera in its X-axis was x″. The variation is then calculated as the difference between x″ and x′. Similarly, the variations in the Y and Z axes may be calculated. Variations in rotational movements may also be calculated in a similar way. For the determined variation in the movement in an axis, the error is ascertained. Subsequently, the average error may be calculated using the ascertained errors for all the axes. Thereafter, the orientation of the axis system of the stereo camera may be rotated to minimize the error with respect to the axis system of the movable platform. Furthermore, scalar multiplications may also be used to minimize errors in each axis.

Precise and constant positioning between the fundus camera and the pupil is one of the essential conditions to capture an image of the highest quality. Any variation in the position due to various factors such as operational conditions during use, environmental factors etc. may affect the quality of the image. In one embodiment of the invention, the position of the fundus camera may be calibrated with respect to the position of the ECT, using an illumination projection. The illumination projection may be the reflections of the illumination source used for flash photography of the fundus. The fundus camera and the illumination source may be mounted on a movable platform. Initially, the centre of the illumination projection of the illumination source may be aligned to the centre of the ECT by using information from the stereo camera. The centre of the ECT may be determined using libraries like OpenCV and methods like morphological operations, filters, contour detection etc. For determining the optimal distance from ECT for fundus imaging, the contour properties of the illumination projections are observed. The contour properties may include the centre of illumination projection, radius and circularity of the projections. The contour properties may also be observed and recorded for components of the illumination projection, where components may be such as reflection of individual Light Emitting Diodes (LEDs) of the illumination source wherein the illumination source may include multiple LEDs. As an example, the most desired properties may be defined as when the circularity of the individual illumination projections of the LEDs of the illumination source is the highest. To achieve the most desired properties of the illumination, the movable platform may be moved in a perpendicular direction to the plane of the ECT. Once the most desired properties are achieved, the optimal coordinates may be captured and saved in the database. The optimal coordinates may be defined as coordinates of the ECT as calculated by the stereo camera. The optimal coordinates also happen to be the coordinates of the pupil where the captured image of the fundus may be of the highest quality. The quality of the fundus images may be determined by sharpness, complete illumination of the retina, gradeability of the fundus image, minimum unwanted reflections etc.

A fundus camera may include various components such as an optical system having several optical lenses, illumination sources etc. The precise positioning of all these components is of utmost importance to capture a fundus image correctly. Due to various reasons such as accidental damages, operational conditions etc. the alignment of the components may change from the desired positions. In one embodiment, the alignment of the centre of illumination projection and the centre of the image sensor of the fundus camera is validated. To validate the alignment, the centre of illumination projection may first be aligned with the centre of the ECT. Subsequently, an image of the ECT is captured using the fundus camera and the centre of the ECT is identified. Further, the error between the centre of the actual image and the centre of the ECT in the fundus image may be calculated. If the calculated error is found within a predefined threshold, the alignment is validated as successful, else a failure.

Alternatively, the validation of the alignment may be performed by first capturing an image of the ECT using the fundus camera. Further, the centre of the ECT is identified. The fundus camera device may now be aligned such that the centre of the ECT matches with the centre of the actual image of the fundus camera. Further, the centre of the illumination projection, as seen in the fundus image, may be identified. The error between the centre of the illumination projection and the centre of the actual image may be calculated. If the calculated error is found within a predefined threshold, the alignment is validated as successful, else a failure.

In one embodiment of the present invention, validation of reprojection error of the markers for a plane may be performed. The calibrated stereo camera may be used to capture the image of a plane of the MCT. For the captured image, object points and the respective image points may be identified for the fiducial markers wherein 2-D coordinates of the object points as printed on the calibration target are known. Further, object coordinates may be defined as 3-D coordinates of the object points with one of the axes set to zero.

Now, the coordinates of the markers may be calculated using the pre-calibrated stereo camera, which may also be referred to as predicted coordinates. For example, a triangulation method may be used to calculate the predicted coordinates from image points. Further, the best-fitting plane may be identified for these predicted coordinates. To calculate the reprojection error, these predicted coordinates may be brought to the same origin and same orientation as the object coordinates. To achieve it, first, the best-fitting plane needs to be rotated in all three axes to match the orientation of the plane of object coordinates. The required rotation in pan and tilt may be calculated by finding the normal to the best-fitting plane of the predicted coordinates. Applying the rotations about the centroid of the predicted coordinates by the calculated pan and tilt may now make the plane of predicted coordinates parallel to the plane of the object coordinates. Further, the predicted coordinates need to be rotated about the axis of the normal to the plane, also referred to as the roll axis here. After applying the roll, the predicted coordinates may align with the object coordinates in all three orientation axes. However, the predicted coordinates are still not in the same plane as the object coordinates and are also displaced in X-Y-Z axes. The centre of the predicted coordinates and centre of the object coordinates may be matched by shifting the centre of the predicted coordinates to the centre of the object coordinates. This helps to achieve the alignment of the object coordinates and the predicted coordinates. Subsequently, the error between the predicted coordinates and the object coordinates can now be calculated by checking the Euclidean distance in X-Y-Z axes between the corresponding coordinates of the markers. Now, a validation of the error is performed to verify if the average error of all the detected markers is less than the threshold value. If the error is more than the threshold value, the stereo camera calibration may be termed as invalid. Further, the stereo camera calibration may be repeated to achieve the reprojection error within the predefined threshold value.

According to one embodiment of the invention, an automatic calibration of the fundus camera device may be performed. The automatic calibration of the fundus camera may be initiated on pushing the start button or through the control unit. Alternatively, the sensors for detection, automatically detect various parameters such as the calibration target, placement of the fundus camera, idle time of the fundus camera device, etc. and may initiate the automatic calibration of the fundus camera device if the fundus camera is placed at position for the automatic calibration. On initiation, all the motors are placed at their home position. Thereafter, a fast validation may be performed to confirm if the existing calibration data are still compatible with the functioning of the fundus camera device. Further, validation of movements and fundus camera image sensor alignment may also be coupled with fast validation. If errors of calibration are below the predefined threshold, the validation may be termed as successful. The calibration target may then be removed, and the fundus camera device may be available for operational use.

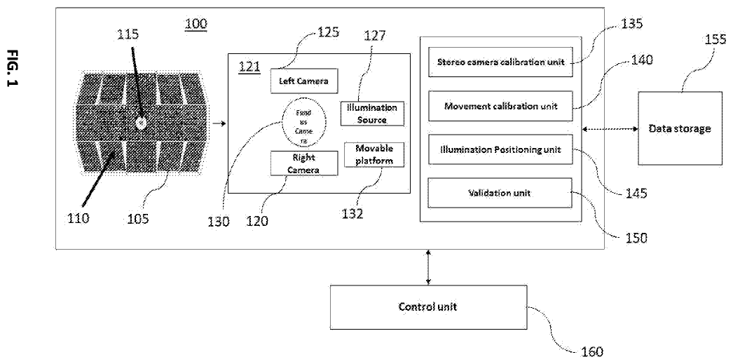

FIG. 1 illustrates a schematic block diagram of an exemplary system for automatic alignment of a fundus camera device according to an embodiment of the present invention. The system 100 may include a fundus camera device 121, MCT (multi-planar calibration target) 110, a stereo camera calibration unit 135, a movement calibration unit 140, illumination positioning unit 145, a validation unit 150, a data storage unit 155 and a control unit 160. Various components of the system 100 may be housed in one housing body or may remain stand-alone units or combinations.

A fundus camera device may include two cameras 120 and 125 of a stereo camera, a fundus camera 130, an illumination source 127 and a movable platform 132. The MCT 110 may comprise multiple planes 105 and an ECT 115. In one example, one or more images of the planes 105 of the MCT 110 may be captured using the stereo camera (120, 125) or the fundus camera 130. The fundus camera 130, the stereo camera (120, 125), and the illumination source 127 may be mounted on a movable platform 132. The stereo calibration unit 135 may be configured to calibrate the stereo camera (120, 125), the movement calibration unit 140 may be configured to calibrate the movement of the movable platform 132, and the fundus camera positioning unit 145 may be configured to calibrate the position of the fundus camera device with respect to the ECT using the illumination projection of the illumination source 127. Further, the validation unit 150 may be configured to validate errors of various components of device 100. Further, the data storage unit 155 may be configured to store calibration data, image data and other associated data in a database. The control unit 160 includes a processing unit, data acquisition unit, motor control unit, illumination control unit, user interface unit, data control unit etc. The control unit is configured to control the movement of the movable platform 132, to control the intensity or power of the illumination sources (not shown in FIG. 1), to acquire the data from the fundus camera 130, the stereo camera (120, 125) and other sensors (not shown). The control unit may further be configured to process and manipulate the data for Stereo camera calibration 135, movement calibration 140, illumination positioning 145, validation 150 and other functionalities part of various embodiments. The control unit may further be used to execute the instructions based on a specific computer readable program.

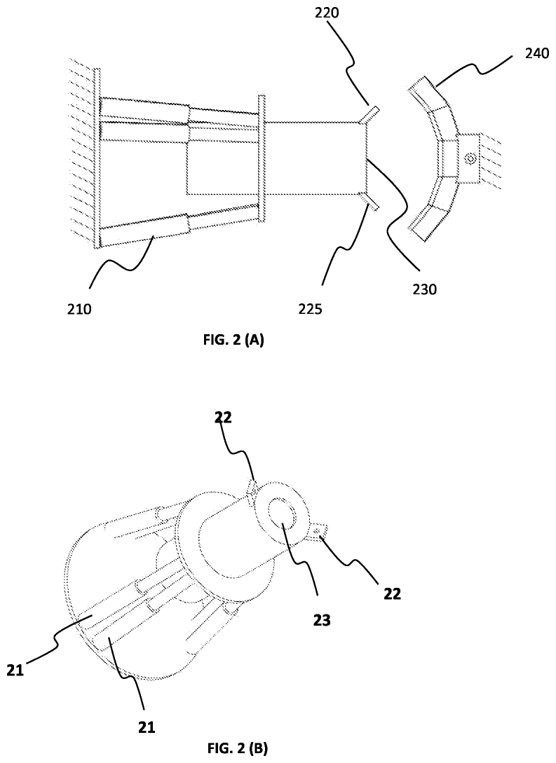

FIG. 2(A) and FIG. 2(B) depict an exemplary projection view and front view of a fundus camera device respectively according to an embodiment of the present invention. FIG. 2(A) depicts a stereo camera (220 and 225), actuators or motors 210, a fundus camera 230 and an MCT 240 mounted on a holder. The stereo camera may include two cameras (220 and 225) which are configured to capture images of the MCT. The cameras (220 and 225) may comprise optical systems to focus and capture images of the intended target. The multiple motors 210 may be configured to actuate the movable platform and manipulate the position of the fundus camera device thereof. The actuation of the motors 210 may be controlled by the control unit 160. The fundus camera 230 includes an optical system. In an example, the optical system of the fundus camera 230 may include multiple lenses, an illumination source, and an image sensor. Further, the fundus camera device may be manoeuvred through the control unit 160 as shown in FIG. 1. FIG. 2(B) shows another view of the fundus camera 230 and the position of the stereo camera (220 and 225) with respect to the fundus camera.

FIG. 3 shows an exemplary multi-planar calibration target according to an embodiment of the present invention. The MCT 315 may comprise multiple planes. Each plane of MCT may be embedded with multiple fiducial markers 320. In one example, Aruco markers may be embedded for identification. Each marker may be assigned a unique identifier. The location of each marker may be identified using object points and mapped image points for the marker. The object points may refer to real-world coordinates and the image points may refer to the pixel coordinate of the marker in the image captured. The ECT 310 may be designed as a dark dot on one of the planes of the calibration target. The size of the dot may be small enough to be detected from the fundus camera, allowing aligning the fundus camera to the ECT. There may be an empty space with no visual disturbances near the ECT. The empty space may be used for illuminating the calibration target and calibrating the positioning of the fundus camera device with respect to the ECT for achieving optimal illumination and imaging of the human eye.

Alternatively, it may also be possible to use fiducial markers such as checkerboard, circles, etc. Such fiducial markers may comprise of only non-unique, only unique or a combination of unique and non-unique features. For example, ChArUco boards are a combination of checkerboard and Aruco markers. Further, partial, or complete views of fiducial markers may also be used in methods explained in the present invention wherein various detecting methods may be used in conjunction to the methods described in this invention. For example, occluded checkerboard detection methods may be used to identify partial views of a checkerboard pattern. Similarly, various techniques may be used to segregate checkerboards by the planes they belong to. Further, object points and image points may be identified in checkerboards by detecting the corners of the checkerboard.

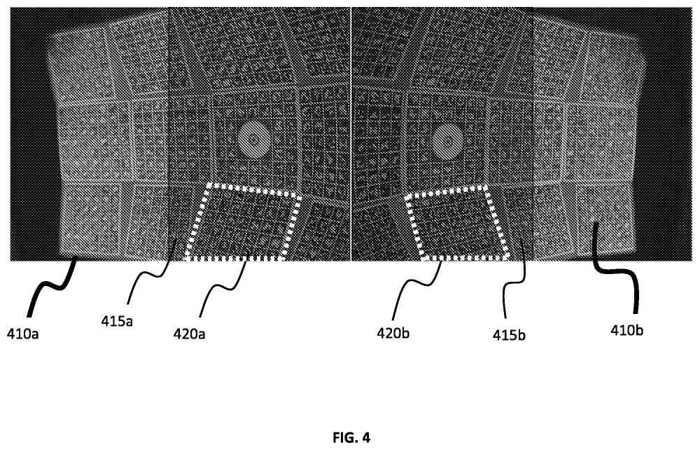

FIG. 4 illustrates images of a calibration target captured by the left and the right camera of the stereo camera according to an embodiment of the present invention. For the same MCT, the view by the left camera and the right camera of the stereo camera is represented by 410a and 410b. In one scenario, the cameras may capture a full view of planes the MCT as represented by dark shaded portions 410a and 410b. In another scenario, the cameras may capture a partial view of a few of the places of the MCT as represented by 420a and 420b. Further, mutually visible sections 415a and 415b may also be identified in the images captured from both cameras. In one embodiment of the present invention, irrespective of a partial or full view of the MCT captured by the cameras, the images may be used to calibrate the camera. On capturing the image, the unique identifiers of the markers and the corresponding object points and the mapped image points may be identified thereof.

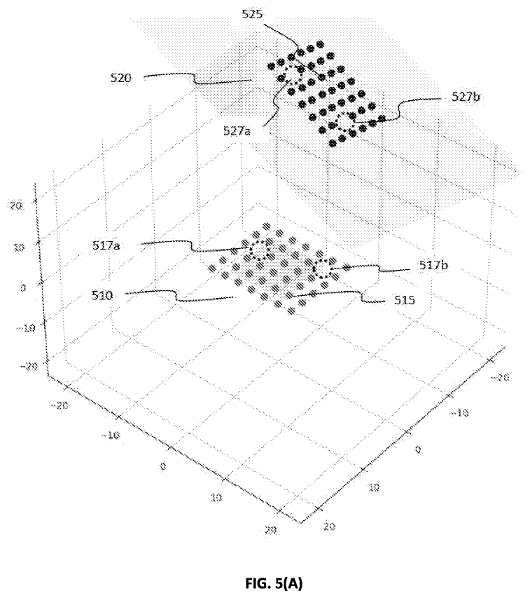

FIG. 5(A), FIG. 5(B), FIG. 5(C), FIG. 5(D), and FIG. 5(E) show exemplary visualizations of reprojection error according to an embodiment of the present invention. An image of one of the planes of the MCT may be captured using a stereo camera. In FIG. 5(A), a plane 510 with object coordinates 515 may be identified corresponding to the markers identified in the captured image. Object coordinates may be defined as 3-D coordinates of the object points with one of the axes set to zero. Further, for example, two markers of the plane 510 may remain unidentified and hence may have unfilled positions in the object coordinates shown in 515. The coordinates of the unidentified markers (517a, 517b) for plane 510 are shown as dotted-line circles. Similarly, the predicted coordinates of the unidentified markers, had they been identified, are shown in 527a and 527b, only for clarity. The technique of the present invention may still be able to calculate the reprojection error even if a few of the markers may remain unidentified. The object coordinates 515 for the markers are pre-known based on the pattern printed on the calibration target. Predicted coordinates 525 may be identified for the markers using a pre-calibrated stereo camera. A best-fitted plane 520, comprising predicted coordinates 525 may be reprojected to the plane 510 of object coordinates 515. To obtain the reprojection error, the predicted coordinates may first be brought to the same origin and the same orientation as the object coordinates.

To obtain the reprojection error, initially, the pan and tilt rotations may be performed for the best-fitted plane 520 as represented by FIG. 5(A). The required rotation in pan and tilt may be calculated by finding the normal to the best-fitting plane of the predicted coordinates. After correcting the pan and tilt of the predicted coordinates 525 with respect to the object coordinates 515, as represented by FIG. 5(B), the predicted coordinates 525 are now projected as shown in FIG. 5(C). Further, the predicted points need to be rotated about the axis of the normal to the plane, also referred to as the roll axis such that the predicted coordinates 525 may be aligned to the object point 515. After performing rotations to the plane 510 in all the axes, the predicted points 525 may be aligned with the object points 515 in all three orientation axes as shown in FIG. 5(D). The best-fitting plane 550 may still not be in the same plane as the plane 560 i.e., the best-fitted plane may be displaced in XYZ axes as shown in FIG. 5(D). After shifting the centroid of the predicted coordinates 525 to the centroid of the object coordinates 515, the predicted coordinates 525 may now be aligned to the object coordinates 515 in both rotation and translation as represented in FIG. 5(E). The error between the predicted coordinates and the object coordinates can now be calculated by checking the Euclidean distance in x-y-z axes between the corresponding coordinates of the markers. If the error is less than the acceptable threshold value, the stereo camera calibration is accepted as a success.

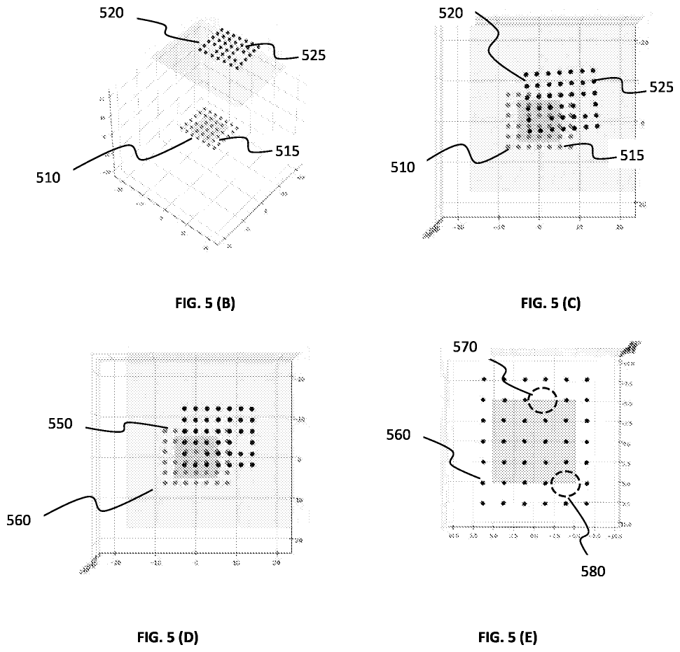

FIG. 6(A), and FIG. 6(B) depicts an exemplary axis system of a stereo camera and a movable platform according to an embodiment of the present invention. The X-axis and the Z-axis of the axis system of the stereo camera are represented by 610 and 620 in FIG. 6(A) while the x-axis and z-axis of the axis system of the movable platform are represented by 630 and 640 in FIG. 6(B). As represented in FIG. 6(A), the axis system of the movable platform and the axis system of the stereo camera may be perfectly aligned. In such a scenario, when the platform moves by say (x, y, z) in its own axis system, the stereo camera observes a movement of exactly (x,y,z) in its own axis system. However, if the axes of the stereo camera and the movable platform don't match, as represented in FIG. 6(B), it may affect the manipulation of the fundus camera device. To match the axis system of the movable platform, the error between the axis orientation of the stereo camera and axis orientation of the movable platform may be identified using movement calibration. To minimize the error, the axis system of the stereo camera may be rotated virtually using the control unit. The virtual rotation of the axis system is performed by the control unit by multiplying a rotation matrix to any predicted coordinates. After applying the rotation, If the error in the movement in the axes of the stereo camera is less than the threshold value, the movement is considered as calibrated, and the data are stored in the database.

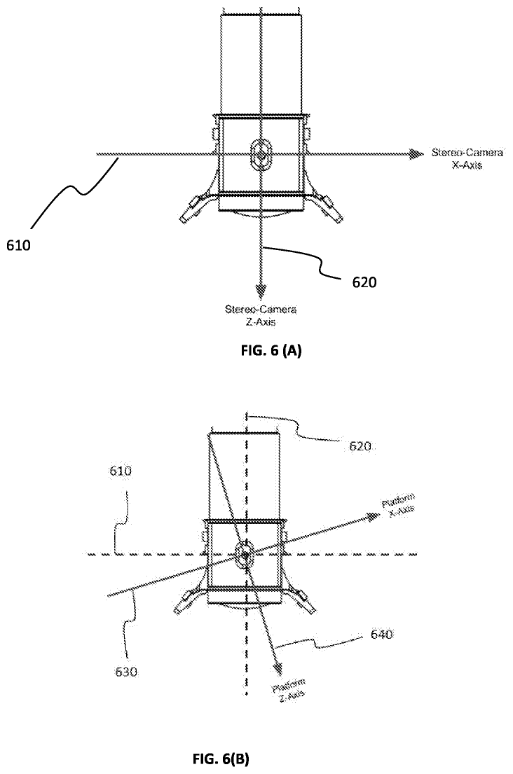

FIG. 7 is a flowchart illustrating a process for automatically calibrating a fundus camera device according to an embodiment of the present invention. The process for automatic calibration may begin with mounting the MCT to its recommended position and starting the automatic calibration process. In one example, the process for automatic calibration may be triggered automatically on automatic detection of a calibration target, idle time, gesture recognition, etc., wherein the sensor or the camera performs the detection. All the motors may be sent to their home positions. At 710, the fast validation of the fundus camera device may be initiated wherein it may be determined if the pre-existing calibration files are still compatible with the functioning of the fundus camera device. The fast validation of the fundus camera device may be provided with the errors in various pre-calibrated components such as stereo camera, movement, illumination positioning, fundus camera image sensor alignment etc. At 720, if the errors for all the components are below a predefined threshold, the fast validation ends with success and the fundus camera device is ready for use. However, if the errors in fast validation are beyond a threshold, then the calibration for the said component such as stereo camera calibration 725, movement calibration for the movable platform 730 or positioning of the fundus camera with respect to the ECT using illumination projection 735 or all said components may need to be performed. Once the calibration for the required components is performed, the fundus camera device is ready for use as represented by 740.

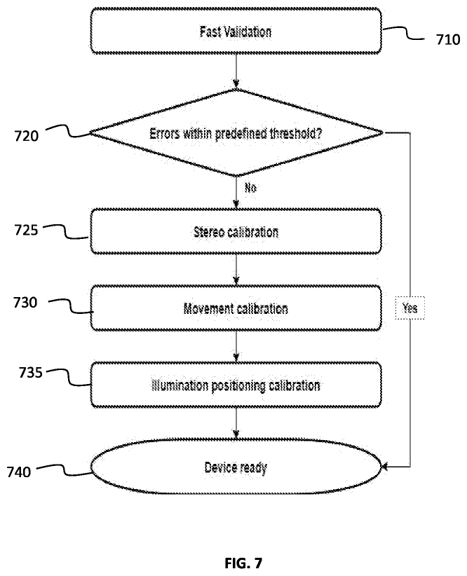

FIG. 8 is a flowchart illustrating a process for calibration of a stereo camera according to an embodiment of the present invention. At 810, the calibration process for the stereo camera may start with capturing multiple images of planes of MCT as shown in FIG. 3. The images of all visible planes may be captured using the left and the right camera of the stereo camera. At 820, the markers may be identified using an identifier. The identifier may be configured to identify all the uniquely identifiable markers visible in the planes. An identifier may include, but is not limited to, a computer-enabled program to detect a marker, scan a marker and the like. At 830, the identified unique markers may be grouped. The grouping of the markers may be performed based on a predefined pattern of the plane to which the markers belong to. For example, markers 0-99 may belong to plane 0 while 100-199 may belong to plane 1. Since images with reduced quality are also accepted in the calibration, a few markers may not be detected due to certain constraints such as focus, mechanical limitations etc. At 840, after segregating the markers for the respective planes, a validation is performed to verify if a plane consists of a minimum number of points acceptable to calibrate the stereo camera. At 850, a corresponding mapping list may be defined with the markers and their known real-world coordinates in 3-D assuming their position in one of the axes to be zero as all the markers belong to the same plane. The mapped image points and object points are stored in the data storage for any further needs such as running world-coordinates reprojection error calculations and the like. At 860, the intrinsic and extrinsic properties of the left and the right camera of the stereo camera are calibrated.

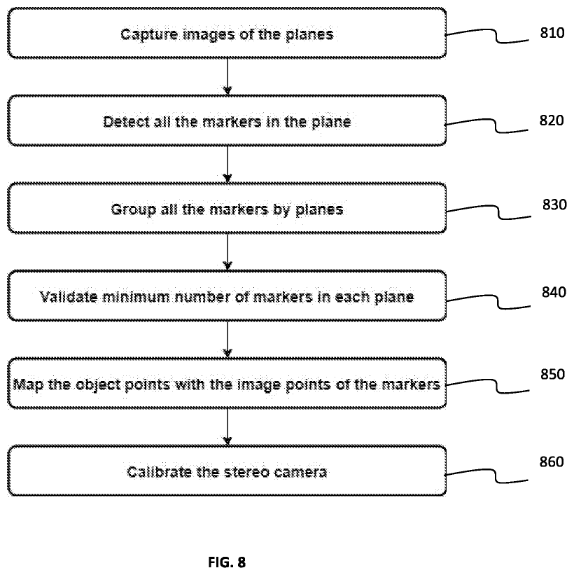

FIG. 9 is a flowchart illustrating a process for movement calibration of a movable platform and a stereo camera according to an embodiment of the present invention. At 910, the axes of the movable platform and the axes of the stereo camera are identified. On selecting a particular axis, any variation in movement in this axis of the stereo camera and the movable platform may be determined at 920. For the determined variation in the movement in an axis, the error is ascertained. Subsequently, at 930, the average error may be calculated using the ascertained errors for all the axes. Thereafter at 940, the orientation of the axis system of the stereo camera may be virtually rotated to minimize the error with respect to the axis system of the movable platform. The virtual rotation of the axis system is performed by the control unit by multiplying a rotation matrix to any predicted coordinates. Furthermore, scalar multiplications may also be used to minimize errors in each axis. Further, it is validated if the average error is less than the predefined threshold. This validation may also be referred to as movement validation. On successful validation, the orientation of the movable platform is calibrated and available for use at 950.

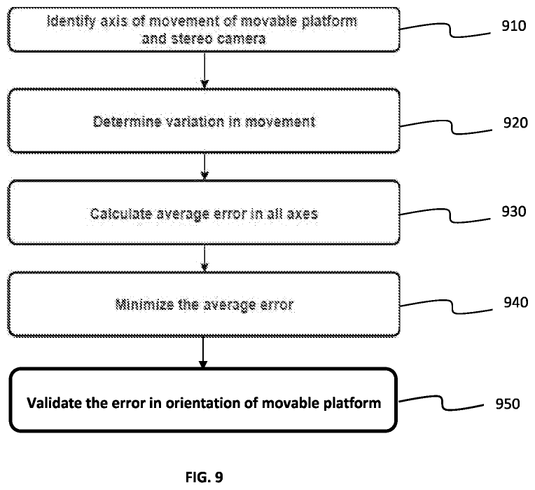

FIG. 10 is a flowchart illustrating a process for calibration of calculation of reprojection error according to an embodiment of the present invention. At 1010, an image of one of the planes of the MCT may be captured using a stereo camera. At 1020, markers are identified using an identifier. At 1030, predicted coordinates of the markers may be calculated using a pre-calibrated stereo camera. A best-fitted plane comprising predicted coordinates may also be identified. Further in 1040, the object coordinates of the markers are also identified. A plane with object coordinates may be identified corresponding to the markers identified in the captured image. Further, a partial view of the planes may also be used where some of the markers of the plane are not identified. The technique of the present invention may still be able to calculate the reprojection error. Further at 1050, the plane comprising predicted coordinates is rotated in all axes to match with the orientation of the plane of the object coordinates. Further at 1060, the predicted coordinates are translated such that the centroid of the predicted coordinates matches with the centroid of the object coordinates. Now at 1070, the error between the predicted coordinates and the object coordinates can be calculated by simply checking the Euclidean distance in x-y-z axes for the respective markers. Further at 1080, an average error may be calculated by summing all the errors and dividing by the number of identified markers. Further at 1090, if the average error is less than the acceptable threshold value, the stereo camera calibration is accepted as a success.

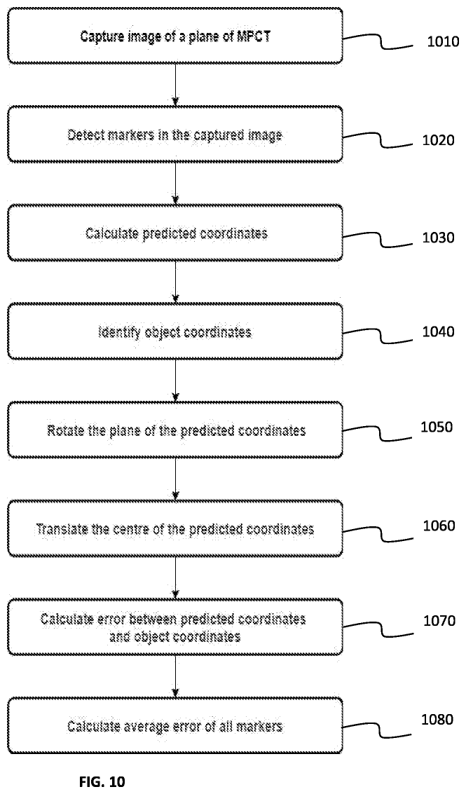

FIG. 11 shows a flowchart of an exemplary process for calibration of the position of the fundus camera device using an illumination source, according to an embodiment of the present invention. In 1110, the fundus camera device may initially be aligned with the ECT in the x-y plane. The X-Y plane may be referred to as the plane parallel to the plane of the ECT. Further in 1120, the fundus camera device is moved in the Z-axis such that the ECT is at an approximate distance of focus, as designed in manufacturing. In 1130, the fundus camera device is moved away from the ECT in the Z-axis by a predefined distance. The predefined distance may be calculated based on the maximum variation in the focus distance of the illumination projection. The variation in the focus distance of the illumination projection may occur due to various factors such as physical damage, operational conditions, mishandling, etc. Further 1140, 1150 and 1160 may be part of an iterative process generally referred to as a loop. In 1140, the fundus camera device may be moved closer to the ECT in the Z-axis by a small step. The small step for movement may be calculated using the resolution of the stereo camera and the optical system of the fundus camera device. Further in 1150, an image of the illumination projection may be captured and properties of the contours of the projections may be calculated and recorded. The image may be captured from the left camera, the right camera, the stereo camera or the fundus camera. At 1160, the steps to obtain the desired properties of the contour properties are repeated till the properties of the illumination projection may start degrading. At 1170, once the properties may start degrading, the loop is exited and in 1170, the coordinates of ECT when the properties of illumination projection were most desirable, are saved as the optimal coordinates. Further, the properties of the contours of the illumination projection may also be saved for use in fast validation or other processes.

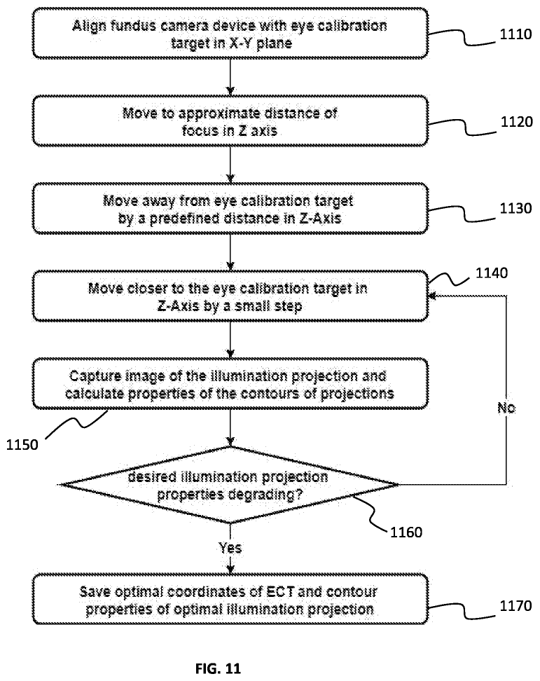

FIG. 12 illustrates a flowchart of an exemplary fast validation process according to an embodiment of the present invention. At 1210, an image of the MCT may be captured using the stereo camera. At 1220, the markers in the captured image may be identified. At 1230, the average of the reprojection error may be calculated for all the visible markers in the captured image. At 1240, the average error may be validated to make sure it is within the predefined threshold. At 1250, the fundus camera device (FCD) may be positioned such that the ECT is at the optimal coordinates. Further, the illumination source may then be turned on and made to illuminate the ECT. Further at 1260, the properties of the contours of the illumination projection may be calculated. At 1270, the error between the saved illumination projection properties of a correctly calibrated fundus camera device and the illumination projection properties observed in this validation may be calculated. Lastly at 1280, if the calculated error in projection properties is within the predefined threshold, validation of position based on illumination may be considered successful. Movement Validation and fundus camera image sensor alignment validation may also be performed during the process of fast validation.

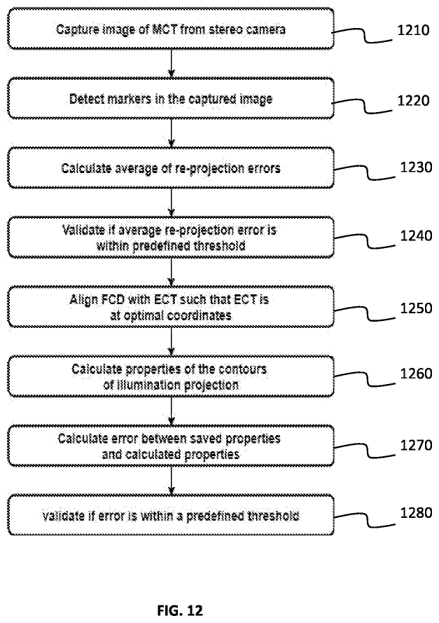

FIG. 13 illustrates a flowchart of an exemplary validation process for the alignment of the centre of the image sensor of the fundus camera according to an embodiment of the present invention. In this example, the alignment of the image sensor may only be validated in the proposed X-Y plane, which is the plane parallel to the plane comprising the ECT. Initially, at 1310, the centre of illumination projection may be aligned with the centre of the ECT. This may be performed by capturing an image of the ECT from any camera that can identify both the illumination projection as well as the ECT during alignment. For example, if the left camera can identify the centre of the illumination projection and the centre of the ECT, the device may be moved in the X-Y plane in small steps until the error observed is zero. Further, at 1320, an image of the ECT may be captured using the fundus camera. At 1330, the centre of the ECT may be determined in the fundus image. At 1340, the error between the centre of the fundus image and centre of the identified ECT may be calculated and at 1350, the calculated error may be validated to make sure it is within the predefined threshold limit. If the error is not within the pre-defined threshold, the fundus images captured during operational use may not be fit to use for application and hence the device may need to be repaired mechanically.

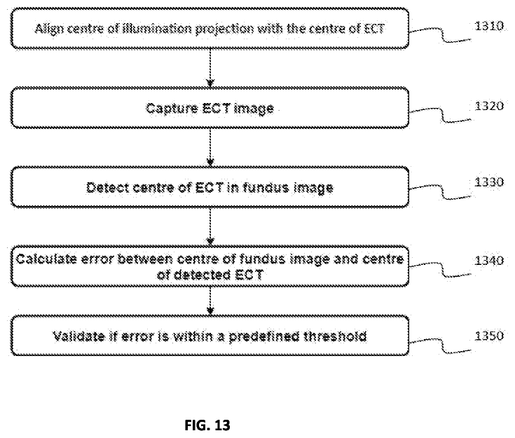

FIG. 14 illustrates a flowchart of an exemplary alternate way of validating the alignment of the centre of the image sensor of the fundus camera according to one embodiment of the invention. At 1410, an image of the ECT may be captured using the fundus camera. At 1420, the fundus camera device may be moved such that the centre of the fundus camera image aligns with the centre of the ECT. At 1430, the centre of the illumination projection may be identified in the fundus image. At 1440, the error between the centre of the illumination projection and the centre of the eye calibration target in the fundus image may be calculated. Further at 1450, the error may be validated to make sure it lies within the threshold limit.

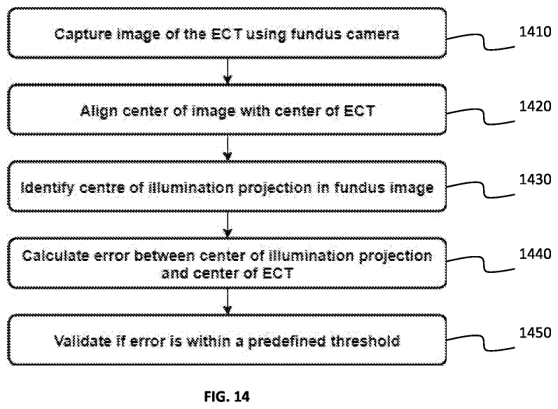

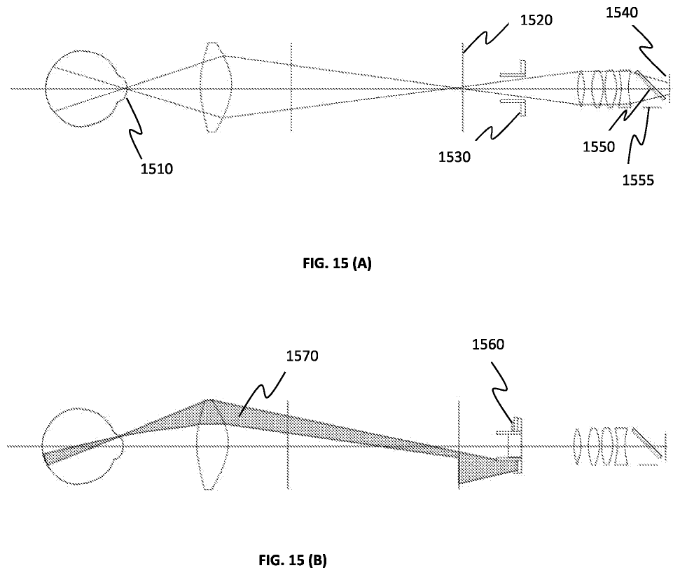

FIG. 15 (A) illustrates an exemplary imaging path of the fundus camera using an optical system according to an embodiment of the present invention. A pupil 1510 of the human eye is depicted for capturing the image. The optical components may further include light masks 1520 and 1530, a beam splitter 150, a gaze target, and an image sensor 1540. Gaze target allows the human eye to be oriented in a particular direction and photograph particular sections of the subject's fundus. The image sensor 1550 may be placed at the rear end of the device to capture images.

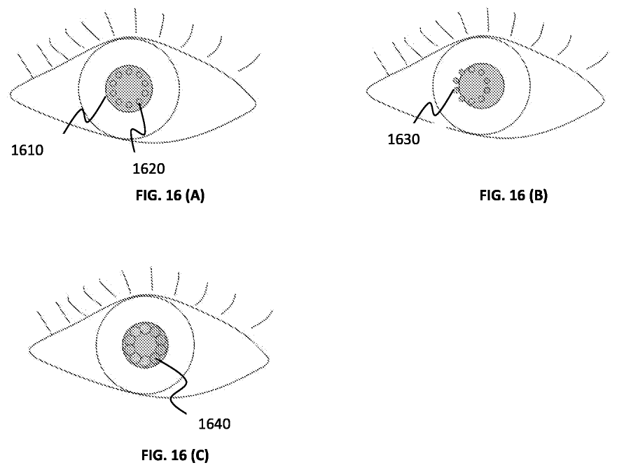

FIG. 15 (B) illustrates an exemplary illumination path of the fundus camera using an optical system according to an embodiment of the present invention. The illumination board 1560 may have one or several LEDs placed in a circular geometry for uniformly illuminating the fundus. The diagram also shows the importance of precise positioning of all the mentioned optical elements required to illuminate and capture images of the subject's eye correctly since a small change in the positioning of any element would cause a drastic change in the path traced by light rays as represented 1570. The slight change in position may affect the illumination and imaging path and therefore the quality of the fundus image as shown in FIG. 16.

FIG. 16(A), FIG. 16 (B), FIG. 16(C) illustrate the exemplary effect of positioning of the fundus camera device to an embodiment of the present invention. FIG. 16 (A) shows a scenario that is an ideal condition where the device is aligned correctly with respect to the subject's eye in all the axes. The eye is properly illuminated, and the desired image of the pupil may be captured. FIG. 16 (B) shows an example where the device is correctly aligned in the Z-axis but not in the XY plane. This results in non-uniform illumination of the eye and may also result in unwanted reflections or a partly illuminated retina in the captured fundus image. Further, in FIG. 16 (C), an example is shown where the device is perfectly aligned in the XY plane, but incorrectly positioned in Z-axis, where the illumination projections are larger and may cause an overlap with the imaging path causing reflections in the captured image.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention.

1. A method of validating alignment of an image sensor of a camera (130) and an illumination projection for use in a system for automatically aligning a camera device (121) which includes the camera (130), wherein a centre of the image sensor is identified; and the camera device (121) also includes a stereo camera (120, 125), in addition to the camera (130), and the camera device (121) includes an illumination source (127) wherein a centre of an illumination projection is identified in an image from the image sensor or from the stereo camera, the method comprising the steps of:
aligning, using the stereo camera (120, 125), the centre of the illumination projection with the centre of an eye calibration target (115) wherein the eye calibration target is included on one of the planes of a multi-planar calibration target (110), wherein each plane of the multi-planar calibration target is embedded with a plurality of fiducial markers, wherein the eye calibration target is a marker, and wherein a size of the eye calibration target is approximately equal to the size of a pupil of an eye;
capturing an image of the eye calibration target using the camera (130) and detecting the position of the eye calibration target;
calculating an error between the centre of the captured image and the centre of the detected eye calibration target; and
validating if the error is within a predefined threshold;
wherein the method further comprises calibration of a position of illumination and the eye calibration target, including steps of:
aligning the centre of the illumination projection to the centre of the eye calibration target;
determining an optimal projection of illumination by moving a movable platform (132) in a perpendicular direction to the plane of a centre of the eye calibration target, wherein the stereo camera, the camera and the illumination source are mounted on the moveable platform; and
identifying optimal coordinates of the eye calibration target for capturing an image by the camera (130) and saving it in a database, wherein the optimal coordinates are the coordinates of the eye calibration target as calculated by the stereo camera, and also the coordinates of the pupil where a captured image of a fundus of the eye is of the highest quality.

2. The method of claim 1, further comprising determining one or more contour properties of the illumination projection, wherein the contour properties are captured when calibration of the optimal position of the camera device with the illumination projection is successfully performed, and wherein an image in which the contour properties are determined is taken from the image sensor or from the stereo camera.

3. The method of claim 2, wherein the contour properties of the illumination projection comprise at least one of centre, radius, and circularity.

4. The method of claim 1, wherein the identifying optimal coordinates step uses the illumination projection in the aligning step.

5. A system comprising means adapted for carrying out all the steps of
a method of validating alignment of an image sensor of a camera (130) and an illumination projection for use in a system for automatically aligning a camera device (121) which includes the camera (130), wherein a centre of the image sensor is identified; and the camera device (121) also includes a stereo camera (120, 125), in addition to the camera (130), and the camera device (121) includes an illumination source (127) wherein a centre of an illumination projection is identified in an image from the image sensor or from the stereo camera, the method comprising the steps of:
aligning, using the stereo camera (120, 125), the centre of the illumination projection with the centre of an eye calibration target (115) wherein the eye calibration target is included on one of the planes of a multi-planar calibration target (110), wherein each plane of the multi-planar calibration target is embedded with a plurality of fiducial markers, wherein the eye calibration target is a marker, and wherein a size of the eye calibration target is approximately equal to the size of a pupil of an eye;
capturing an image of the eye calibration target using the camera (130) and detecting the position of the eye calibration target;
calculating an error between the centre of the captured image and the centre of the detected eye calibration target; and
validating if the error is within a predefined threshold;
wherein the method further comprises calibration of a position of illumination and the eye calibration target, including steps of:
aligning the centre of the illumination projection to the centre of the eye calibration target;
determining an optimal projection of illumination by moving a movable platform (132) in a perpendicular direction to the plane of a centre of the eye calibration target, wherein the stereo camera, the camera and the illumination source are mounted on the moveable platform; and
identifying optimal coordinates of the eye calibration target for capturing an image by the camera (130) and saving it in a database, wherein the optimal coordinates are the coordinates of the eye calibration target as calculated by the stereo camera, and also the coordinates of the pupil where a captured image of a fundus of the eye is of the highest quality.

6. A computer program comprising instructions for carrying out all the steps of a method, when said computer program is executed on a computer system,
the method being a method of validating alignment of an image sensor of a camera (130) and an illumination projection for use in a system for automatically aligning a camera device (121) which includes the camera (130), wherein a centre of the image sensor is identified; and the camera device (121) also includes a stereo camera (120, 125), in addition to the camera (130), and the camera device (121) includes an illumination source (127) wherein a centre of an illumination projection is identified in an image from the image sensor or from the stereo camera, the method comprising the steps of:
aligning, using the stereo camera (120, 125), the centre of the illumination projection with the centre of an eye calibration target (115) wherein the eye calibration target is included on one of the planes of a multi-planar calibration target (110), wherein each plane of the multi-planar calibration target is embedded with a plurality of fiducial markers, wherein the eye calibration target is a marker, and wherein a size of the eye calibration target is approximately equal to the size of a pupil of an eye;
capturing an image of the eye calibration target using the camera (130) and detecting the position of the eye calibration target;
calculating an error between the centre of the captured image and the centre of the detected eye calibration target; and
validating if the error is within a predefined threshold;
wherein the method further comprises calibration of a position of illumination and the eye calibration target, including steps of:
aligning the centre of the illumination projection to the centre of the eye calibration target;
determining an optimal projection of illumination by moving a movable platform (132) in a perpendicular direction to the plane of a centre of the eye calibration target, wherein the stereo camera, the camera and the illumination source are mounted on the moveable platform; and
identifying optimal coordinates of the eye calibration target for capturing an image by the camera (130) and saving it in a database, wherein the optimal coordinates are the coordinates of the eye calibration target as calculated by the stereo camera, and also the coordinates of the pupil where a captured image of a fundus of the eye is of the highest quality.
